(19) United States
(12) Patent Application Publication
(10) Pub. No.: US 2021/0275287 A1
(43) Pub. Date: Sep. 9, 2021

(54) EXPANDER OF ORTHOTOPIC ARTIFICIAL BLADDER ENDOPROSTHESIS

(71) Applicants: Bianca DORDONI, Cremona (IT); Bianca SAMBUSSETI, Cremona (IT); Elisa SAMBUSSETI, Cremona (IT)

(72) Inventor: Antonio SAMBUSSETI, (US)

(21) Appl. No.: 17/255,147

(22) PCT Filed: Jun. 27, 2019
(86) PCT No.: PCT/EP2019/067217

§ 371 Date: Dec. 22, 2020

(30) Foreign Application Priority Data
Jun. 29, 2018 (IT) 102018000006814

(51) Int. Cl. A61F 2/04; A61B 90/00

(52) U.S. Cl. CPC A61F 2/042; A61B 90/02; A61F 2210/0076; A61F 2250/0098; A61F 2230/0093; A61F 2250/0003; A61F 2230/0071

(57) ABSTRACT

A description is given of a deformable and expandable element, or expander, for internal support for an orthotopic artificial bladder endoprosthesis in a textile of biodegradable biocompatible material, characterised in that the expander has a substantially hollow shape, made in medical grade silicone elastomer, and provided with a conduit for injecting in its interior a fluid so as to change from a non-expanded configuration to an expanded configuration, the conduit being integral with a one-way check valve applied on the expander.

### BACKGROUND OF THE INVENTION

### Field of the Invention

The present invention relates to an expander (i.e. expandable element) for orthotopic artificial bladder endoprosthesis, made of bioabsorbable and biocompatible textile such as PGA (polyglycolic acid) textile or the like.

### Description of the Related Art

As is known, when a patient's bladder is affected by serious incurable diseases that compromise its proper functioning, it is desirable to completely replace it with an artificial bladder prosthesis (orthotopic endoprosthesis).

Orthotopic artificial bladder endoprostheses are known that are substantially spherical in shape, made in biocompatible and/or biodegradable material such as, for example, PGA fibre textile, in order to completely replace the natural bladder. See for example the artificial bladder in PGA textile, in the form of a cuff, described in WO2016/051333.

To ensure that its shape is maintained during the growth of the new bladder on the cuff during the decomposition of the textile in PGA (resorption), the use is provided of an expandable element, placed inside said endoprosthesis in PGA textile: said element, in its expanded form, acts as a support for the cuff so as to prevent the collapse of the cuff under the weight of the growing new cellular textile.

The expandable element described in WO2016/051333 consists of a series of arms that can be switched from a retracted configuration, required when inserting said element into the endoprosthesis during the surgical operation, to an extended configuration required during the phase of creation of the new bladder.

However, the use of an expander element in Nitiol results in a high cost, also in light of the structural complexity of the movement of the arms that compose it, which are fixed to a rod in such a way as to take on a C shape after bringing the ends of said rod closer together.

WO2016/051333 discloses an orthotopic artificial bladder endoprosthesis made in PGA, which internally contains an expandable element which is equipped with a valve for the introduction and extraction of a filling fluid for the element.

### SUMMARY OF THE INVENTION

The object of the present invention is to overcome, at least in part, the disadvantages of the prior art by providing an expander for an orthotopic artificial bladder endoprosthesis in PGA fibre textile or similar materials, that is effective, economical and simple to manufacture.

These and other objects are achieved by the expander according to the invention having the features listed in the appended independent claim 1.

Advantageous embodiments of the invention are disclosed by the dependent claims.

An object of the present invention relates to a deformable and expandable support element, here also defined as “expander” for the sake of simplicity, to be housed inside an orthotopic artificial bladder endoprosthesis formed by a cuff in PGA fibre textile, wherein said expander is a spherical or substantially spherical soft element, internally hollow, capable of changing from a retracted or non-expanded configuration (at rest, in a non-use condition) to an expanded configuration (when in use) by means of the injection of a physiological solution.

Said cuff, made in a PGA fibre textile, may have the same or similar construction features to those described in the patent application WO2016/051333 in the name of the Applicant, incorporated herein in its entirety for reference.

Said spherical or substantially spherical expandable element is made in soft medical grade silicone and is covered at least externally with a pyrolytic turbostratic carbon film, although it is preferable for the expander to be completely covered with this film.

The expander according to the invention for an orthotopic artificial bladder endoprosthesis is then formed by an internally hollow sac or balloon, made with a medical grade silicone membrane, for example an elastomeric silicone formed of copolymers of dimethylsiloxane and methylvinylsiloxane, preferably a mixture of poly(dimethylsiloxane) with vinyl end-groups (PDMS) and vinyl-methylsiloxane-dimethylsiloxane copolymer with trimethylsiloxane end-groups, e.g. MED 4750 silicone or MED 4735 silicone, marketed by the firm Nusil Technology.

Said silicone membrane can be obtained advantageously by dipping, a technology already known in the art and described for example in the patent application WO2007/039159 in the name of the Applicant, incorporated herein in its entirety for reference.

It is understood that other techniques known in the art, such as moulding, may be used to make the medical silicone expandable sac or balloon in accordance with the present invention, without departing from the scope of the present invention.

In particular, medical silicone can advantageously comprise radio-opacifying additives such as barium sulfate, titanium dioxide and the like, so as to make the silicone radio-opaque and allow the detection of the expander with the techniques normally used such as X-rays, ultrasound, and other analysis systems, if necessary.

Said spherical or substantially spherical expander element is also provided with a check valve that has a conduit or channel through which to inject a fluid, e.g. a liquid such as physiological saline solution, so as to expand said spherical element to the desired and/or suitable volume to sustain said cuff in PGA textile.

Said spherical element, also denoted here by the term expander, typically has a volume of about 300 cc in non-expansion conditions (at rest, when not in use) but can expand to up to 600 cc in volume, after the injection of saline.

### BRIEF DESCRIPTION OF THE DRAWINGS

Further features of the invention will be made clearer by the detailed description that follows, referring to a purely non-limiting example of embodiment illustrated in the accompanying drawings, in which:

FIG. 1 is a view from above of the expander according to the present invention for an orthotopic artificial bladder endoprosthesis in PGA textile;

FIG. 2 is a partially interrupted vertical section view of the expander of FIG. 1;

FIG. 3 is an enlarged view of the part enclosed in the circle of FIG. 2 including a check valve;

FIGS. 4a, 4b, 4c are vertical section views of three different check valves similar to the one shown in FIG. 3;

FIG. 5a is a view from above of the precursor of the expander of FIG. 1;

FIG. 5b is a vertical section view of the precursor of the expander of FIG. 1.

### DESCRIPTION OF THE PREFERRED EMBODIMENTS

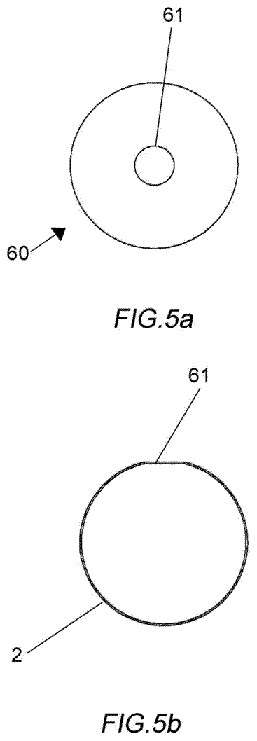

Referring to FIGS. 5a-5b, a precursor of an expander is illustrated according to the present invention, denoted in the abovementioned drawings by reference numeral 60.

Said precursor 60, which is in the form of a balloon or sac, consists of a multilayer membrane 2 made in soft and elastic silicone so as to be a deformable and expandable element.

It should be noted that said membrane 2 can be made in a single layer without thereby departing from the scope of the present invention.

Preferably, membrane 2 of balloon 60 has a total thickness of about 600-800 microns.

In a preferred embodiment, the membrane 2 of the balloon 60 consists of twenty layers of silicone, each with a thickness of about 30 microns.

In particular, the multilayer membrane 2 is obtained through a processing procedure called dipping: by means of a machine called in fact dipping equipment, a balloon or sac is created, internally hollow, starting from a single layer of silicone, and then superimposing, on each layer, other layers until the desired overall thickness is obtained.

More specifically, this multi-layer dipping technique consists in making the first layer, making it evaporate with cyclohexane for 10 minutes, superimposing the second layer, making it evaporate it with cyclohexane for 10 minutes, and so on up to the last layer.

At this point, the stratified silicone membrane 2, which is in a semi-fluid state, is placed in an oven for vulcanization, at a temperature of about 150° C. and for a time ranging from 30 min to 1 h, depending on the size of the expander that is to be made.

After the vulcanization cycle, the multilayer silicone membrane 2 is in its optimal softness and elasticity consistency, and no longer in a semi-fluid state.

In a preferred embodiment said precursor 60 has a diameter of around 85 mm (when the volume is 300 cc).

Subsequently, at least on the outer surface of the precursor 60, a pyrolytic turbostratic carbon microfilm or layer with a thickness of approximately 0.2-0.3 micron is applied.

The deposition of the pyrolytic turbostratic carbon microfilm on the outer surface of precursor 60 takes place according to a known technique, for example by physical vapour deposition (PVD), in particular by the physical deposition technique known as “sputtering”, or by arc deposition, preferably by sputtering.

At this point a circular through hole 61 is made at the head of said precursor 60: said hole 61 is intended to be closed/covered by a check valve provided with an injection channel and with an upper portion in the form of an opened umbrella as will be explained here below in detail.

In a preferred embodiment, said hole 61 has a diameter of about 20 mm.

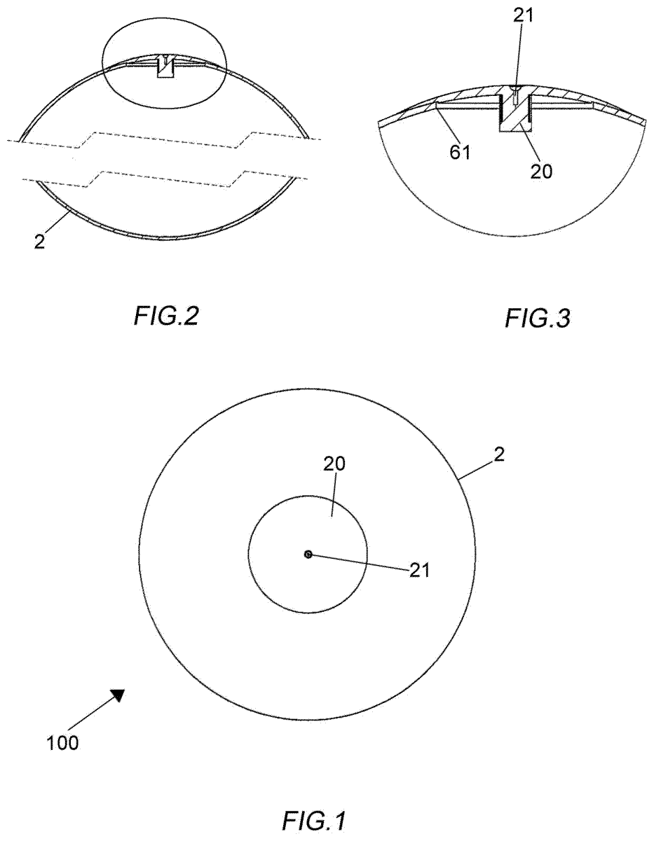

Once the hole 61 has been obtained on the precursor 60, a check valve denoted in FIGS. 1-4 by reference numeral 20 will be applied.

Said check valve 20 is an umbrella valve.

The check valve 20 (one-way check valve) can be made of metal or silicone, generally medical one, preferably silicone (silicone check valve).

In a particularly preferred embodiment, the check valve 20 is an umbrella silicone valve.

Said check valve 20 is glued with silicone glue to balloon 60 to cover hole 61.

Said check valve 20 has a conduit or channel 21 which is integral with said check valve 20, through which to inject a physiological saline solution into the expander 100, in use, so as to inflate/expand said expander 100 up to the pre-set volume that is apt to make said expander 100 act as an internal support for the cuff in PGA textile (not shown in the drawings).

Said physiological solution can be injected by means of a syringe or similar means.

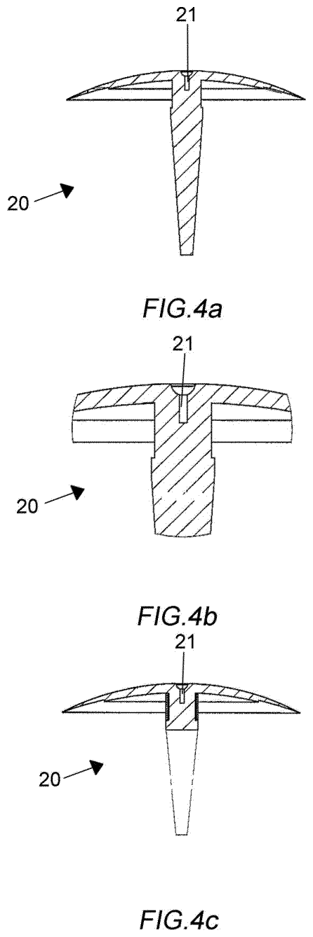

Said conduit 21 is formed in the body of the valve 20 as shown in FIGS. 4a, 4b, 4c, at the central axis of said valve 20: said umbrella valve 20 is therefore particularly efficient since the duct 21 also acts as a guide for the syringe needle which remains centrally positioned, avoiding accidental tilting and piercing the balloon during the inflation phase.

Moreover, this type of valve has reduced number of components that form it and is suitable to be placed in small spaces.

The element thus obtained is the expander that is the object of the present invention, denoted overall by reference numeral 100 in FIG. 1.

The expander 10 is therefore substantially in the form of a balloon both in unexpanded form (i.e. precursor 60) and in expanded form.

In use, once the patient's natural bladder has been removed, the orthotopic artificial bladder endoprosthesis (cuff) in PGA textile (not illustrated) is implanted and contains the expander 100 of the present invention, in non-expanded form.

The PGA textile cuff, in which the present expander 100 has to be housed, is provided with appropriate connectors to connect said cuff to the ureters and urethra by means of resorbable sutures, such as for example described in patent application WO2016/051333, incorporated herein in its entirety by reference.

Once the connections with the ureters have been made, by means of resorbable sutures, the expander 100 of the present invention, is brought into the extended/expanded configuration: to do this, the surgeon injects a saline solution with a syringe inserted in the conduit 21 of the check valve 20, so as to inflate the expander 100 up to the desired volume to act as an internal support for said cuff.

After which, the third and last connector is fixed to the urethra with a resorbable suture and the operating field is closed.

At this point, it is necessary to wait for the predetermined period in order to allow the reconstruction of the new bladder.

After this period, generally around 1-2 months, the surgeon operates surgically through a mini-cystotomy (at the end of the regeneration of the new autologous bladder textile), piercing the expander to make it take on the retracted configuration, and subsequently pulling it out through the urethra: in this way further invasive surgery is avoided, saving the patient a further hospital stay.

In fact, the resorbable textile cuff and the deformable support element of the present invention are made independently one from the other and no special actions and precisions are required.

Numerous detail changes and modifications may be made to the present embodiment of the invention, within the reach of a person skilled in the art, in any case coming within the scope of the invention disclosed by the appended claims.

1. An orthotopic artificial bladder endoprosthesis in a textile of biodegradable biocompatible material comprising an inner support constituted by a deformable and expandable element, or “expander”, (100) apt to act, when in expanded form, as inner support,
wherein said expander (100) is substantially in the form of a balloon, internally hollow, made in medical-grade silicone, and provided with a conduit (21) for injecting into an interior of the expander, by means of a syringe, a liquid so as to change from a non-expanded configuration to an expanded configuration,
said conduit (21) being formed in, and integral with, a one-way check valve (20) applied on said expander.

2. The endoprosthesis according to claim 1, wherein at least the outer surface of said expander element (100) is covered with a microfilm of pyrolytic turbostratic carbon.

3. The endoprosthesis according to claim 1, wherein said silicone of said expander element (100) is elastomeric and comprises copolymers of dimethylsiloxane and methylvynilsiloxane.

4. The endoprosthesis according to claim 1, wherein said medical-grade silicone of said expander element (100) comprises radio-opacity additives.

5. The endoprosthesis according to claim 1, wherein said expander element (100) has a volume of approximately 300 cc in a non-expanded condition, and a volume up to 600 cc in an expanded condition.

6. The endoprosthesis according to claim 1, wherein said expander element (100) is formed of a multilayer membrane (2).

7. The endoprosthesis according to claim 1, wherein said expander element (100) has a thickness of about 600-800 microns.

8. The endoprosthesis according to claim 1, wherein said check valve (20) of said expander element (100) is an umbrella valve.

9. The endoprosthesis according to claim 8, wherein said check valve (20) of said expander element (100) is made of metal or silicone.

10. The endoprosthesis according to claim 2, wherein at least the outer surface of said expander element (100) is covered with a microfilm of pyrolytic turbostratic carbon with a thickness of about 0.2-0.3 micron.

11. The endoprosthesis according to claim 9, wherein said check valve (20) of said expander element (100) comprises a silicone check valve.

12. The endoprosthesis according to claim 9, wherein said check valve (20) of said expander element (100) is a silicone umbrella check valve.

13. An expander element (100) substantially in the form of a balloon, internally hollow, made in medical-grade silicone, and provided with a conduit (21) for injecting into an interior of the expander, by means of a syringe, a liquid so as to change from a non-expanded configuration to an expanded configuration,
said conduit (21) being formed in, and integral with, a one-way check valve (20) applied on said expander.

14. The endoprosthesis according to claim 1, wherein said silicone of said expander element (100) is elastomeric and comprises MED 4735™ silicone and MED4750™ silicone from the firm Nusil Technology.

15. The endoprosthesis according to claim 4, wherein radio-opacity additive is selected from a group consisting of barium sulphate and titanium dioxide.

16. The endoprosthesis according to claim 2, wherein said expander element (100) has a volume of approximately 300 cc in a non-expanded condition, and a volume up to 600 cc in an expanded condition.

17. The endoprosthesis according to claim 3, wherein said expander element (100) has a volume of approximately 300 cc in a non-expanded condition, and a volume up to 600 cc in an expanded condition.

18. Endop The endoprosthesis rosthesis according to claim 4, wherein said expander element (100) has a volume of approximately 300 cc in a non-expanded condition, and a volume up to 600 cc in an expanded condition.

19. The endoprosthesis according to claim 5, wherein said expander element (100) has a volume of approximately 300 cc in a non-expanded condition, and a volume up to 600 cc in an expanded condition.

20. The endoprosthesis according to claim 2, wherein said expander element (100) is formed of a multilayer membrane (2).
